(19) United States
(12) Patent Application Publication
(10) Pub. No.: US 2024/0311699 A1
(43) Pub. Date: Sep. 19, 2024

(54) AUTOMATED PARALLELIZED PROCESSING OF DECISION-TREE GUIDELINES USING ELECTRONIC RECORD

(71) Applicant: ROCHE MOLECULAR SYSTEMS, INC., Pleasanton, CA (US)

(72) Inventors: Charles Alcorn, Oakland Park, FL (US); Alexander Wu, Fremont, CA (US); Wei Yao, Foster City, CA (US); Ju Zhang, Sunnyvale, CA (US)

(73) Assignee: ROCHE MOLECULAR SYSTEMS, INC., Pleasanton, CA (US)

(21) Appl. No.: 18/674,661

(22) Filed: May 24, 2024

Related U.S. Application Data
(63) Continuation of application No. PCT/US2022/050958, filed on Nov. 23, 2022.
(60) Provisional application No. 63/285,685, filed on Dec. 3, 2021.

(51) Int. Cl. G06N 20/00

(52) U.S. Cl. CPC G06N 20/00

(57) ABSTRACT

A machine learning model for traversing a decision tree, the machine learning model trained from a structured data set including a first set of key-value pairs and subject-specific criteria using the key-value pairs. The first set of key-value pairs is transformed into a second set of key-value pairs, which are projected to a subject-specific point within a multi-dimensional space. The decision tree includes decision and leaf nodes. Each leaf node is connected to a root node via a leaf-node-specific trajectory. Each decision node corresponds to a criterion using a value in the second set of key-value pairs. For each leaf node, a leaf-node-specific point within the multi-dimensional space is determined using the leaf-node-specific trajectory, and a similarity score is determined using the leaf-node-specific and subject-specific points. A subset of the leaf nodes is identified using the scores. State or protocol information for each leaf node in the subset is retrieved.

### CROSS-REFERENCE TO RELATED APPLICATIONS

This application is a continuation of and claims priority to PCT Application No. PCT/US2022/050958, filed on Nov. 23, 2022, which claims priority to U.S. Provisional Application No. 63/285,685, filed on Dec. 3, 2021, is hereby incorporated by reference in its entirety for all purposes.

### BACKGROUND

Protocols are frequently used to address a particular issue or to attempt to avoid or postpone a subsequent undesired outcome. However, selecting a protocol for a particular use case can be challenging, as the likelihood of a protocol being effective frequently depends on current states and event history.

For example, suppose that a protocol is to be used to protect a computing system from security threats. Efficacy of a given protocol may depend on the type of data that is stored on the computing system, the type of network(s) to which the computing system is connected, whether the computing system was previously accessed without authorization, etc. As another example, suppose that a protocol is to be used to inhibit progression of a disease of a particular human subject. Efficacy of a given protocol may depend on which disease sub-type the subject has, a current level of disease progression, and demographics of the subject.

Further, selecting a protocol frequently involves considering a combination of different states and a combination of different prior events in combination. Different decision-makers may then arrive at different protocol selections.

Further yet, different types and amounts of data may be available in various instances to inform selection of a protocol. Thus, it can be particularly challenging to identify a technique that can reliably transform variable sizes and types of input data into a useful output.

In some industries, guidelines are used in an attempt to remove noise and variability across protocol selections and to facilitate consistent and interpretable protocol selections. However, some guidelines (e.g., clinical practice guidelines) are particularly complex (e.g., including protocol elements that-when printed-span hundreds of cross-referenced pages) and/or change frequently as new information becomes available. Thus, it can be difficult for a human decision-maker to navigate the guidelines efficiently, and the frequent changes can make it expensive to implement via software. For example, a change in an upstream consideration or decision may affect how each of some or all downstream decisions are to be made. (Consider an instance where a consideration is changed from characterizing whether an age of a computing system or subject is within one or two age groups to whether the age is within one or four age groups. All four age groups may then be associated with different subsequent considerations relative to those of the original two age groups.) The complexity and frequent changes can result in the guidelines being infrequently used.

Thus, it would be advantageous to identify and use a technique that facilitates more efficient and more consistent selection of protocols.

### SUMMARY

A computer-implemented method is provided for using transformations and projections to predict a state or effective protocol. A structured data set is accessed that includes a first set of key-value pairs. Each of the first set of key-value pairs characterizing an assessment result or protocol characteristic for a subject. The first set of key-value pairs is transformed into a second set of key-value pairs, wherein at least some keys in the second set of key-value pairs are different from each key in the first set of key-value pairs. The second set of key-value pairs are projected to identify a subject-specific point within a multi-dimensional space. One or more decision trees are accessed that include a plurality of decision nodes and a plurality of leaf nodes. Each of the plurality ofleafnodes is connected to a root node via a leaf-node-specific trajectory. Each of the plurality of decision nodes corresponds to a criterion based on at least one value in the second set of key-value pairs. For each leaf node in the one or more decision trees, a leaf-node-specific point within the multi-dimensional space is determined based on the leaf-node-specific trajectory. For each leaf node in the one or more decision trees, a similarity score is determined based on the leaf-node-specific point and the subject-specific point. An incomplete subset of the plurality of leaf nodes is identified based on the similarity scores. State information or protocol information associated with each leaf node in the incomplete subset is retrieved. An output is generated that is associated with the subject that includes the state or protocol information.

For each leaf node in the one or more decision trees, determining the leaf-node specific point may include: transforming the leaf-node-specific trajectory into a first leaf-node-specific data set using text extraction; transforming the first leaf-node-specific data set into a leaf-node-specific set of key-value pairs; and projecting the second leaf-node-specific set of key-value pairs to identify the leaf-node-specific point.

Determining the similarity score may include applying a cosine similarity function.

The method may further include: for each term of a set of terms, determining an inverse trajectory frequency that indicates how frequently the term occurs across leaf-node-specific trajectories associated with leaf nodes in the one or more decision trees and/or determining a term frequency that indicates how frequently the term occurs in each leaf-node-specific trajectory; where values in the second set of key-value pairs are defined based on the determined inverse trajectory frequencies and/or the term frequencies.

The output may further include, for each node in the incomplete subset, queries represented by decision nodes in the leaf-node-specific trajectory.

The output may include protocol information that identifies a potential treatment for the subject.

The structured data set may include, for each of the first set of key-value pairs, an initial timestamp, and transforming the first set of key-value pairs into a second set of key-value pairs may include: classifying a particular key-value pair of the first set of key-value pairs as an indexing event; generating, for each key-value pair of the first set of key-value pairs, a modified timestamp using the initial timestamp associated with the key-value pair and the initial timestamp associated with the particular key-value pair; detecting that a decision node in the one or more decision trees includes a query to determine whether a particular event occurred within a particular time period relative to occurrence of another particular event, wherein the other particular event corresponds to the indexing event; performing a query to determine whether the first set of key-value pairs includes a first particular key-value pair that is representative of the particular event and that is associated with a modified timestamp within the particular time range; and defining a second particular key-value pair based on a result of the query, wherein the second set of key-value pairs includes the second key-value pair.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by some embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows a computing network 100 for transforming decision trees to facilitate more efficient processing and updates and using the transformed decision trees according to some embodiments of the invention.

FIG. 2 shows an exemplary process for transforming decision trees and data sets by generating and using a multi-dimensional space to identify predicted state or protocol information according to some embodiments of the invention.

FIGS. 3A and 3B illustrate select portions of an exemplary decision tree.

FIG. 4 illustrates a corresponding portion of an exemplary unstructured data set of a subject.

FIGS. 5A-5C show exemplary parallels between variables corresponding to data that is available in exemplary subject data (e.g., that is used to generate structured subject data) and variables that are pertinent to queries that are identified within decision nodes in exemplary decision trees.

FIG. 6A illustrates a node in a decision tree referring to (linking to) two Principle pages.

FIG. 6B illustrates the information corresponding to a structured data set and mapped information pertaining to transformed navigation to a decision tree.

FIG. 6C illustrates criteria in two exemplary decision-tree paths.

FIG. 7 shows exemplary performance metrics characterizing accuracy of results generated using projections in a multi-dimensional space.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

In some embodiments, a decision tree in a set of guidelines is transformed by detecting terms that differentiate between distinct trajectories in the decision tree. Then each trajectory and/or leaf node in the decision tree can be represented as a point within a multi-dimensional space, where the different dimensions correspond to different terms (e.g., which may include key-value pairs, keys from key-value pairs, conditions, or potential results of evaluating one or more conditions) and where a value for each dimension indicates how frequently the term is present in the corresponding trajectory. A use-case-specific data set (e.g., corresponding to an individual subject or system) can include a set of key-value pairs, and these pairs may be transformed to map to a use-case-specific point within the multi-dimensional space. Further, each leaf node in the decision tree (which may include a node representing a potential outcome of a decision but that does not necessarily represent a termination of a trajectory) may be projected into the multi-dimensional space. Distances between the use-case-specific point and each of the points associated with a leaf node can be determined, and the use case can be assigned to the leaf node associated with the shortest distance. Information corresponding to the leaf node can then be retrieved and output.

This approach thus avoids iterative and time-consuming processing of queries in the decision tree. The transformation technique additionally facilitates handling of data sets with missing values. Rather than being “stuck” at a decision node due to missing value, the applicable corresponding query can essentially be ignored or considered in the alternative. Further, by avoiding the iterative approach of navigating a decision tree, code that is defined to identify a leaf node for a given use case can be easily updated in response to detecting a change in the decision tree, without needing to unwrap series of nested conditioned statements.

### II. Exemplary System for Parallelized Processing of Decision Trees

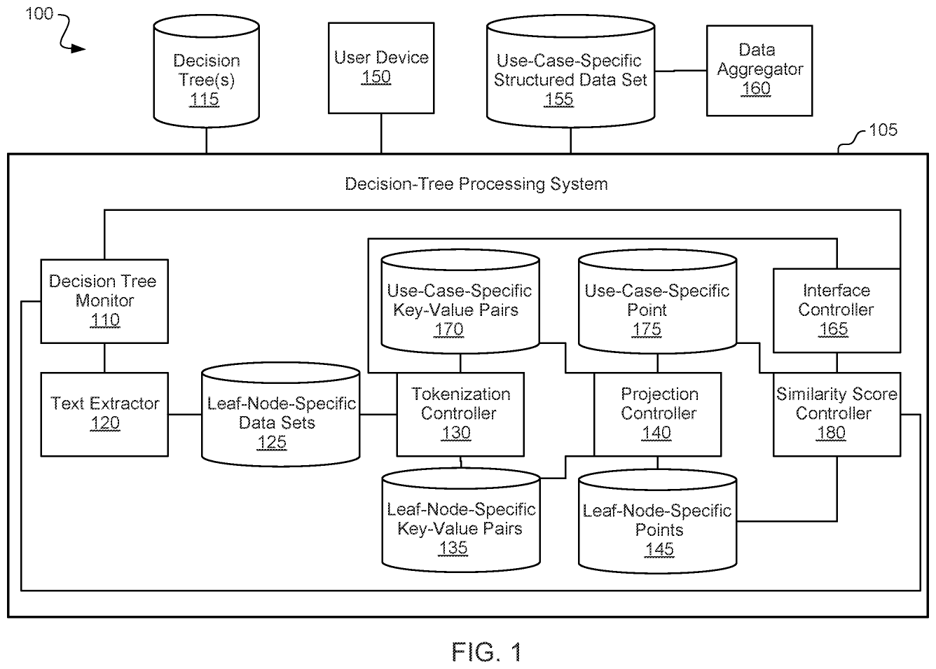

FIG. 1 shows an exemplary network 100 for transforming decision trees to facilitate more efficient processing and updates and using the transformed decision trees. The network 100 includes a decision-tree processing system 105 that can include computing system (e.g., a cloud computing system, a server, one or more computers, etc.). The decision-tree processing system 105 includes a decision tree monitor 110 that accesses one or more decision trees 115. The decision tree monitor 110 may access the decision tree(s) 110 by (for example) scraping data from a webpage that includes the decision tree(s) 110, downloading a file that includes the decision tree(s), or receiving an electronic message (that includes the decision tree(s) 115) from another computing system.

The decision tree(s) 115 include a set of decision nodes and a set ofleaf nodes. Each decision node includes a query. Each leaf node represents a particular protocol or a particular predicted state. For example, a leaf node can represent a predicted current security risk in a computing system or a predicted state of a subject's medical condition. As another example, a leaf node can represent a protocol that includes one or more particular actions that may be performed to facilitate securing a computing system or treating a condition.

Each decision-node query can request a value for a variable that pertains to (e.g., informs) a prediction or recommendation in the guidelines. Each decision-node query can involve an assessment of one or more terms and/or one or more subject-specific key-value pairs (e.g., each of which may correspond to a given term and a particular but associated value). The assessment may include determining whether each of, which of, or any of, one or more criteria are satisfied.

In some instances, the leaf nodes in a given decision tree 115 are unique relative to each other, though multiple trajectories may connect a root node to a given leaf node. For example, each leaf node may be associated with information identifying a different recommended protocol (e.g., treatment) though a given leaf node may be connected to a root node via multiple series of potential query responses. In some instances, each leaf node in the decision tree(s) is connected to a root node via only one trajectory, such that each leaf node corresponds to a specific set of query responses. In this case, at least some of the leaf nodes may be associated with the same information (e.g., and may correspond to a recommendation of a same protocol).

A text extractor 120 extracts text from the decision tree(s) 115 so as to detect the queries in the decision node. For each unique leaf node, the text extractor 120 defines a leaf-node-specific data set 125 to include one or more text strings that identify the queries and responses that were associated with a trajectory connecting a root node and the leaf node.

While some words (or numbers or other character strings) may contribute to the differentiation between different leaf-node-specific data sets 125, others may not. For example, all leaf-node-specific data sets 125 may include many uses of the word “the”, meaning that detecting the word “the” provides no information as to which leaf-node-specific data set 125 is being assessed. Meanwhile, detecting a word string that indicates that a particular fourth-line treatment has previously been administered may provide substantial information as to which leaf-node-specific data set 125 is being assessed.

Thus, a tokenization controller 130 can collectively process the leaf-node-specific data sets 125 corresponding to multiple leaf nodes in the decision tree to identify the degree to which various terms differentiate between the data sets. The processing can include determining, for each term and each leaf-node-specific data set, how many times the term appeared in the data set. This quantity may (but need not) be normalized based on the total number of terms in the leaf-node-specific data set to define a term frequency. A term may include a single word, multiple words, a text string (that includes or that lacks one or more spaces), etc. Tokenization controller 130 can be configured to break down long text into words and phrases. The processing can further include determining, for each term, an inverse document frequency that is based on within how many different leaf-node-specific data sets the term appeared. The inverse document frequency may be defined as a log of the total number ofleaf-node-specific data sets divided by the quantity of leaf-node-specific data sets within which the term appeared. A vectorization controller (not shown) may generate an interim score to each term-trajectory pair based on the term frequency and the inverse document frequency. For example, the interim score may be the product between the term frequency in the leaf-node-specific data set 125 and the inverse document frequency. A score may further be defined for each term as (for example) the maximum of the interim scores associated with the term (across trajectories). In some instances, a vectorization controller (not shown) uses the term frequency-inverse document frequency technique for assigning a term score to each term

In some instances, supervised learning is used to facilitate identifying the terms that support predictions as to which leaf node a trajectory will connect from a decision node. For example, a set of records may be labeled (e.g., by humans or by using codes) so as to indicate that evaluation of a decision node-based on the records-would result in moving from the decision node to a given next node. To illustrate, the set of records may include a report characterizing one or more CT scans, and a label may indicate a partial or full trajectory of a decision tree (e.g., ending at a node corresponding to to a recommended subsequent action), whether a number of tumors has exceeded a predefined threshold. Labels may be identified by human reviewers manually or assisted by computer. Various techniques (e.g., supervised learning) may be used to iteratively improve label prediction. Labels may indicate whether the report indicates to which stage of cancer a given current progression corresponds (e.g., where various branches from the decision node correspond to different stages of progression) or an extent to which a given disease has progressed in a particular time period. A machine-learning model (e.g., a natural language model) may then use various reports and labels to identify one or more select features, tokens, and/or key-value pairs that are informative as to how to evaluate the decision of the decision node.

In instances where the decision tree is particularly large, it may be difficult to obtain sufficient labels to support fully supervised learning. This data issue is amplified by the fact that trajectory directions corresponding to individual decision nodes may be unbalanced. To illustrate, a given decision node may correspond to 99.99% of instances that proceed in one direction as compared to 0.01% that proceed in another direction. To ensure that the model is not biased, it may be important to secure sufficient samples corresponding to the other direction and/or to apply a balancing technique.

The extent to which this is possible and/or the cost (e.g., the time, resource, and/or financial cost) of this approach can compound, as there may be multiple unbalanced decision nodes throughout layers in the decision node. Therefore, it may be advantageous to focus on labeling efforts. The focus may include (for example) prioritizing an underrepresented trajectory label and/or prioritizing one or more underrepresented features. In some instances, the focus may include prioritizing one or more (e.g., a set of) underrepresented features that are associated with one or more trajectories to lead to an underrepresented intermediate node or an underrepresented edge node. For example, an feature-importance technique may be applied to identify the extent to which various decision nodes influence trajectory outputs (e.g., in view of underlying branching probabilities and/or data availability) and/or the imbalance previously observed for or predicted for the decision nodes. Therefore, a prioritization may be set that predicts an importance of securing context-specific labels corresponding to underrepresented pathway labels.

In some instances, the tokens can be identified based on the learned features that are important for decision-node advances. In some instances, training may be implemented that utilizes (for example) the tokens corresponding to the context-specific labels (e.g., and potentially other data). These predictions may be used to predict values that were missing from input data sets and/or so as to predict a partial or full remaining portion of a trajectory corresponding to a given instance.

Tokenization controller 130 can define a key for each term assigned a term score that exceeds or meets a predefined threshold (e.g., an absolute or relative threshold). For each leaf node, tokenization controller 130 can then define a leaf-node-specific set of key-value pairs 135 that includes or represents each defined key and that includes (for each key) a corresponding value that indicates whether the key was detected in the corresponding leaf-node-specific data set. It will be appreciated that “a value” that corresponds to a key or token may include (for example) a particular category, a particular numerical value, a value within a particular range, a category within a particular group of categories, or a value that satisfies a given condition (where the condition may potentially further depend on a value corresponding to another key or variable). Tokenization controller 130 breaks down each text string in a decision-tree path (or of subject data) into tokens (e.g., n-grams, which can include one word, two words, etc.). The value in the key-value pair may be defined to identify how many times or how frequently (across terms) the key was detected in the text associated with the leaf node. It will be appreciated that the key may correspond to any of one or more words, numbers, symbols, etc. For example, a key-value pair may indicate that a number of lesions detected in a scan of a subject collected within the last week is between 70%-90% of the number of lesions detected in a scan obtained by imaging the same subject 2-3 weeks ago. Thus (for example), a token may be identified based on supervised or unsupervised learning, where the token may identify one or more particular variables and one where one or more conditions (e.g., involving one or more thresholds or matches) to which the token pertains. The key may identify that a variable of interest for the given decision node pertains to the relative number of lesions, and the value may identify the precise relative number of lesions (or a given range within which the precise relative number of lesions falls).

A vectorization controller (not shown) can store the set of values in each of the leaf-node-specific set of key-value pairs 135 as a leaf-node-specific point 145 in a multi-dimensional space, where one dimension represents different decision-tree trajectories and another dimension represents different keys. Because keys may have been defined by prioritizing selecting terms that were in only a subset of the leaf-node-specific data sets 125, the leaf-node specific points 145 may be rather separated from each in the multi-dimensional space. In some instances, the leaf-node-specific point 145 is defined to include scaled versions of the set of values in the leaf-node-specific set of key-value pairs 135, where the scaling factor applied to each value is based on (e.g., positively correlated with) the term score associated with the corresponding key. In some instances, a leaf-node-specific point 145 may include at least one value that corresponds to or represents a range or category. In this instance, the leaf-node-specific point 145 may correspond to an area, volume, or sub-space within the multi-dimensional space.

A user device 150 can send a communication to the decision-tree processing system 105 to request that one or more leaf nodes and/or information associated with one or more leaf nodes be identified for a particular use case. For example, the request may correspond to a request to identify any leaf node associated with a present or recent time point for the subject or any leaf node associated with a probability of occurrence that exceeds a predetermined threshold for the subject (e.g., within a given time period or at a given future time point). The particular use case may correspond to a particular subject at a particular point in time. For example, if each leaf node is associated with information that identifies a particular treatment protocol, the user device 150 may request an identification of a top 5 treatment recommendations for a given subject at a present time.

The request may include a use-case-specific structured data set 155 or may include information as to how to access the use-case-specific structured data set 155. The use-case-specific structured data set 155 may include a set of key-value pairs that correspond to the particular use case (e.g., a particular subject). The use-case-specific structured data set 155 may include one or more log messages (e.g., that includes the key-value pairs). The use-case-specific structured data set 155 may include part or all of an electronic health record that includes timestamped and sequentially ordered key-value pairs (e.g., in one or more log messages) that convey (for example) demographics, past diagnoses, any current diagnosis, recent symptoms, laboratory results, imaging results, vital signs, professional assessments, hospitalizations, etc. of a subject. For each of one, more or all keys in the use-case-specific structured data set 155, the key is potentially not present in the leaf-node-specific key-value pairs 135.

Data in the use-case-specific structured data set 155 may have been initially provided from multiple sources. A data aggregator 160 may have collected the data from these sources and structured the data (e.g., using predefined keys).

An interface controller 165 of decision-tree processing system 105 may receive the request from user device 150 and use-case-specific structured data set 155. Tokenization controller 130 can then transform use-case-specific structured data set 155 to use-case-specific key-value pairs 170, which include the same keys as those used in the leaf-node-specific key-value pairs 135. Because the use-case-specific structured data set 155 can already include key-value pairs, searching for particular terms may be insufficient to detect whether a given data set includes information pertaining to a given key that had been defined using the decision tree(s) tree 115. Rather, a mapping and/or look-up table may be used to determine-for each key that had been defined by tokenization controller 130—how to determine the corresponding value using the use-case-specific structured data set 155. In some instances, the mapping may be a one-to-one mapping where the keys that are mapped are identical or are representing the same type or very similar types of data. In some instances, the mapping relates a single key that had been defined by tokenization controller 130 to multiple keys in the use-case-specific structured data set 155. For example, a key of “malignant lymphoma” may be mapped to ((Hodgkin Lymphoma: YES) OR (“Non-Hodgkin Lymphoma: YES) AND (Malignant: YES)), thereby relating to three keys in the use-case-specific structured data set 155. After the tokens are identified, to identify the mapping, the tokenization controller 130 may use a conversion look-up table to associate a key defined by the tokenization controller 130 with one or more keys in the use-case-specific structured data set 155 and to identify any logic and/or math that is to be used to transform corresponding values.

In some instances, the mapping relates to evaluating timestamps in the use-case-specific structured data set 155. Evaluating timestamps may include defining an index date (e.g., as a date on which a particular diagnosis was made) and modifying other timestamps to be relative to the index date. Then if a key is defined as representing whether a given event occurred (e.g., a given treatment was received or a given stage of a disease had been reached) within a time period relative to a diagnosis date, tokenization controller 130 can determine whether a the use-case-specific structured data set 155 includes a log message that identifies a code corresponding to the event that is associated with a modified timestamp that is less than the duration of the time period.

The projection controller 140 can store the set of values in the use-node-specific set of key-value pairs 170 as a use-case-specific point 175 in the multi-dimensional space. In some instances, the values are scaled based on a distance between the use-case specific point 175 and a leaf-node specific point.

The similarity score may be based on and/or may be negatively correlated with a distance between the points. For example, the similarity score may be the reciprocal of the distance, the negative of the distance, or a constant minus the distance.

In some instances, the similarity score and/or the distance is generated using a cosine similarity score, a correlation, or another comparative metric.

The similarity score controller 180 can use the similarity scores to select an incomplete subset of the leaf nodes for the request. The incomplete subset can correspond to leaf nodes associated with the highest similarity scores relative to other leaf nodes in the decision tree(s) 115. The incomplete subset can be defined as the leaf nodes corresponding to similarity scores that exceed an absolute or relative similarity-score threshold. For example, the incomplete subset can include leaf nodes corresponding to then highest similarity scores (where n is predefined or specified by a user).

The decision tree monitor 110 can retrieve information corresponding to each leaf node in the subset. The information can include (for example) a name of the leaf node, metadata of the leaf node, and/or content cited in the leaf-node representation (e.g., content from a leaf-node linked file or document). The information can include a predicted current state (e.g., a predicted stage of a disease or a predicted level of security against malicious threats) or a recommended protocol (e.g., treatment plan or security enhancement) to implement. For example, a protocol may identify a composition or active agent; a dosage; and/or a schedule (indicating when the composition or active agent is to be administered).

The interface controller 165 can transmit the information retrieved for the subset of leaf nodes (or a processed version thereof) to the user device 150. The interface controller 165 may further identify key-value pairs associated with each of the subset of trajectories. Thus, a user may be able to identify which key-value pairs led to a given predicted state or a given protocol identification. The identification may identify leaf-node-specific key-value pairs and may also identify any departures in such pairs in the use-case-specific key-value pairs 170. Thus, even if a user's key-value pairs fail to match each key-value pair in a trajectory, any trajectory departure can be conveyed. Further, this automated facilitates non-iterative processing, which saves substantial time over traditional techniques for traversing decision nodes and saves a tremendous amount of time over humans attempting to wade through complex decision trees (e.g., and resolve unclear decisions).

### III. Exemplary System for Parallelized Processing of Decision Trees

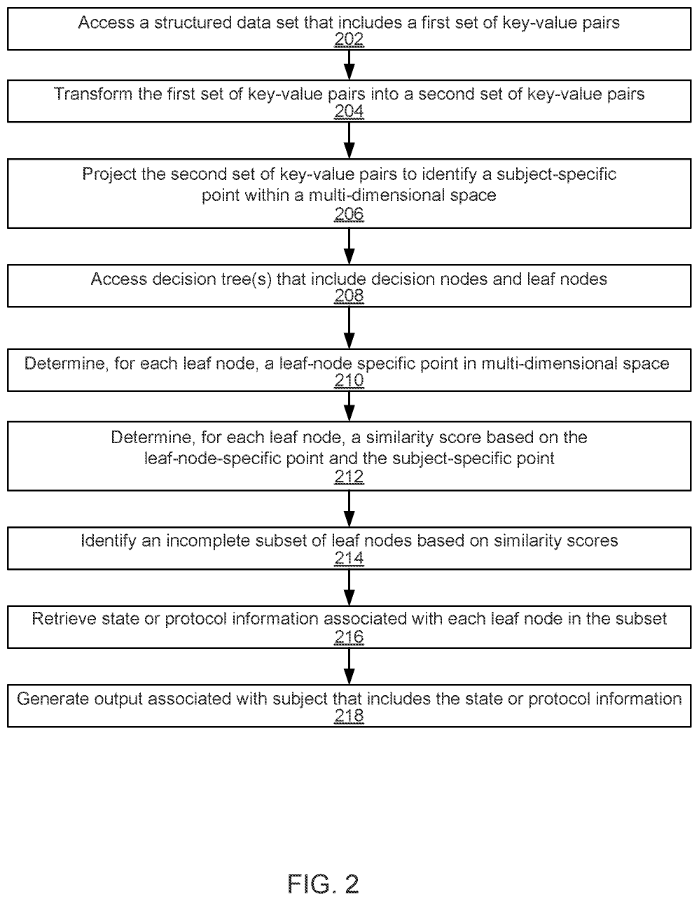

FIG. 2 shows an exemplary process for transforming decision trees and data sets by generating and using a multi-dimensional space to identify predicted state or protocol information according to some embodiments of the invention.

At block 202, the interface controller 165 accesses a structured data set that includes a first set of key-value pairs. The structured data set can correspond to a particular structure (e.g., including keys from a predefined set of keys, having key-value pairs present in a particular order, and/or being in a log file). The structured data set can be use-case-specific structured data set .

At block 204, the tokenization controller 130 transforms the first set of key-value pairs into a second set of key-value pairs. Each of at least some keys in the second set of key-value pairs may be different than any key in the first set of key-value pairs. The transformation can include determining a given second key-value pair of the second set of key-value pairs by (for example) identifying a single corresponding value in the first set of key-value pairs; performing a calculation using multiple values in the first set of key-value pairs; or performing a conversion of a single corresponding value in the first set of key-value pairs (e.g., to map a numeric value to a range or the converse, to identify a negative of a value, to convert units, etc.).

Keys of the second set of key-value pairs may have been identified by tokenization controller 130 to include variables that—alone or in combination-are informative at distinguishing between different trajectories in one or more decision trees. The scores of the second set of key-value pairs may have been selected by using TF-IDF to highlight terms that are represented significantly higher in frequency in an incomplete subset ofleaf-nod trajectories than in others.

At block 206, the projection controller 140 projects the second set of key-value pairs to identify a subject-specific point within a multi-dimensional space. For example, the subject-specific point can include some or all of the values in the second set of key-value pairs. As another example, each of one, more or all of the values in the second set of key-value pairs may be scaled (e.g., based on a TF-IDF value associated with the corresponding key), and the subject-specific point can include the scaled values.

At block 208, the decision tree monitor 110 accesses one or more decision trees. Each of the one or more decision trees includes a set of decision nodes and a set of leaf nodes. The decision tree(s) can correspond to one or more guidelines (e.g., regarding how to diagnosis or treat a particular subject).

At block 210, the projection controller 140 determines, for each leaf node in the set(s) of leaf nodes, a leaf-node specific point in the multi-dimensional space. For example, text from the decision tree(s) can be extracted and a set of leaf-node-specific key-value pairs can be identified. The key-value pairs can include keys that were identified by tokenization controller 130 to include variables that—alone or in combination—are informative at distinguishing between different trajectories in one or more decision trees. The keys of the leaf-node-specific key-value pairs may have been selected by using n-gram tokenization. TF-IDF may be utilized to calculate values in key-value pairs to indicate the extent to which a given term appears significantly higher in frequency than in others. Some or all of the keys of the leaf-node-specific key-value pairs may be the same as or may correspond to keys of the second set of key-value pairs.

At block 212, the similarity score controller 180 determines, for each leaf node in the set(s) of leaf nodes, a similarity score based on the corresponding leaf-node-specific point and based on the subject-specific point. The similarity score may be a numeric value along a predefined scale. The similarity score may be determined based on or may include a distance between the subject-specific point and the leaf-node-specific point in the multi-dimensional space. Additionally or alternatively, the similarity score may be determined based on or may include a cosine similarity score between the subject-specific point and the leaf-node-specific point in the multi-dimensional space.

At block 214, the similarity score controller 180 identifies an incomplete subset of the set(s) of leaf nodes in the decision tree(s) based on the similarity scores. In instances where a decision tree includes multiple trajectories intersecting with or ending at a single leaf node, identifying an incomplete subset can include identifying an incomplete subset of trajectories in the decision trees.

The incomplete subset of leaf nodes can include one or more leaf nodes.

Identifying the incomplete subset can include identifying each leaf node (or trajectory) associated with a similarity score that exceeds a predefined or user-selected absolute or relative threshold. For example, the incomplete subset can include leaf nodes associated with a similarity score above 90% or 0.9. As another example, the incomplete subset can include leaf nodes associated with the top 4 similarity scores across leaf nodes.

At block 216, the decision tree monitor 110 retrieves state information or protocol information associated with each leaf node in the subset of the set(s) of leaf nodes. For example, the state information can identify a particular disease, disease stage, disease sub-type, disease-progression, responsiveness to a given class of treatment, etc. As an additional or alternative example, the protocol information can identify a recommended treatment (e.g., therapy). The treatment identification may identify a particular active ingredient, composition, route of administration, dosage and/or schedule. The information may be retrieved from (for example) a source of the decision tree(s), a source of a file or information that was used to generate the decision tree(s), an external source, or an internal source. The information may be retrieved using (for example) a look-up function.

At block 218, the interface controller 165 generates an output associated with the subject, where the subject includes the state information or the protocol information or a processed version thereof. The output may further include information about the corresponding trajectory/trajectories leading to the leaf node(s) in the subset. For example, for each decision node in a given trajectory, the output may identify the query and the corresponding value that was evaluated (or whether any corresponding value was even available to evaluate).

The output can then be transmitted (e.g., via a webpage or electronic communication) to the user device.

### IV. Examples

### IV.A. Example 1

Exemplary Challenges of Evaluating Decision Trees using Structured Subject Data

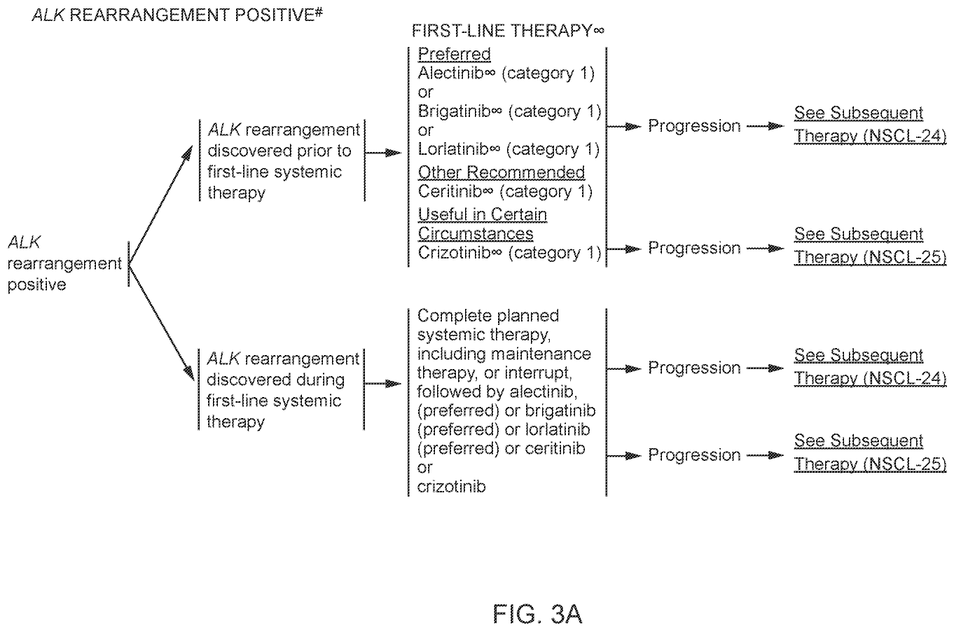

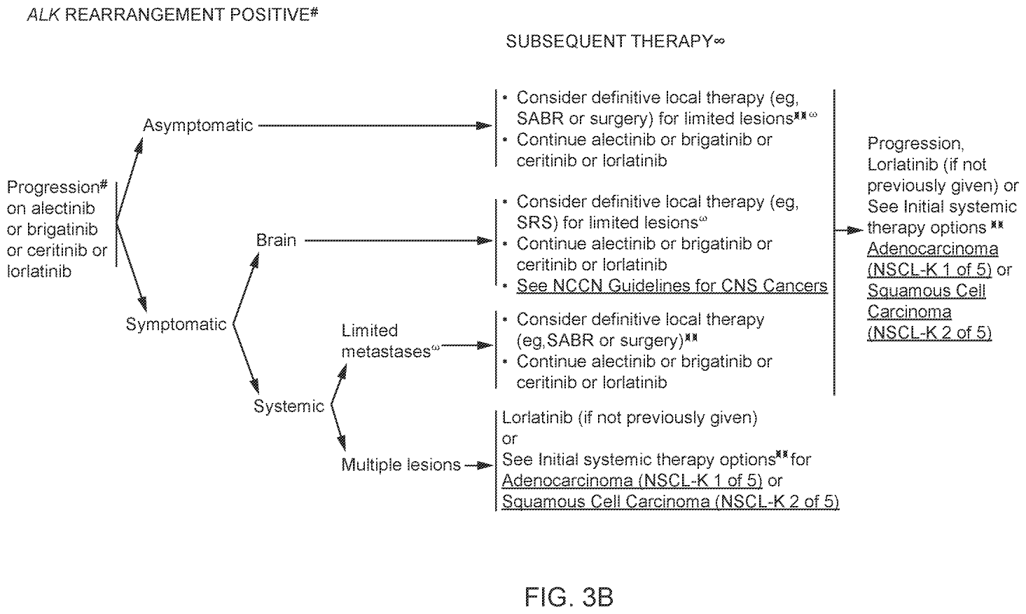

FIGS. 3A and 3B illustrate select portions of a decision tree, and FIG. 4 illustrates a corresponding portion of an unstructured data set of a subject. In FIG. 3A, a current traversal of the decision tree has indicated that a particular gene mutation (ALK rearrangement) has been detected. The first decision to be made in the traversal of the depicted portion of the decision tree is whether this mutation was discovered before a first-line systemic therapy was administered or during such administration. If the rearrangement was discovered before the therapy, the tree traversal proceeds to a decision node to identify which first-line therapy was administered. Meanwhile, if the rearrangement was discovered during the therapy, the tree traversal proceeds to a decision node to identify which therapy was administered after complete planned systemic therapy. Next decision nodes include a query as to whether progression occurred.

When ALK rearrangement was discovered prior to the first-line systemic therapy; the first-line therapy used was one of Alectinib, Rigatinib, Lorlatinib or Certinib; and progression occurred, traversal of the decision tree proceeds to the portion of the decision tree shown in FIG. 3B. The next query to be evaluated is whether the subject is symptomatic or symptomatic. If the latter, it is to be determined whether the symptoms are brain symptoms or systemic. If system, a next query is to characterize the symptoms as limited metastases or multiple lesions.

While at least some of the responses to the queries of decision nodes represented in FIG. 3A and FIG. 3B can be determined using the subject's unstructured data set shown in FIG. 4, determining these responses using an automated approach is challenging. For example, the structured data set is longitudinal. The longitudinal data can also be diverse in terms of variables and size. Identifying a protocol to reliably transform such diverse data in a meaningful way can be particularly challenging. Further, identifying the temporal relations of events requires pulling from different fields to align various events with dates and/or temporal-order information. Further, some of the data that is requested by the decision tree (e.g., whether symptoms are systematic or brain symptoms) is not available and must be inferred. The structured data set further is missing data (e.g., see the biomarker testing dates). Further yet, as noted above, the depicted example of subject data is unstructured. While protocols can be implemented to convert the data to structured data, attempting to determine which variables are important to track (given the complexity of decision trees and the frequent changes of the same) is difficult and does not address problems pertaining to missing data and the challenges with determining the relative timing of various types of events.

Given that the subject data and decision tree may refer to different keys or labels, evaluating a query that depends on the decision tree's structure may depend on an ability to map information in the subject data to variables identified in the decision tree. FIG. SA shows two examples of how subject data may be mapped to decision-tree variables based on differences between how concepts are represented. For example, “Adenocarcinoma, Large Cell, NOS” is synonymous with “Non-squamous cell”. As another example, the depicted example illustrates how structured data may include data (e.g., “BRAF”) that is a different representation or at a different level of precision (e.g., “BRAFV600E”).

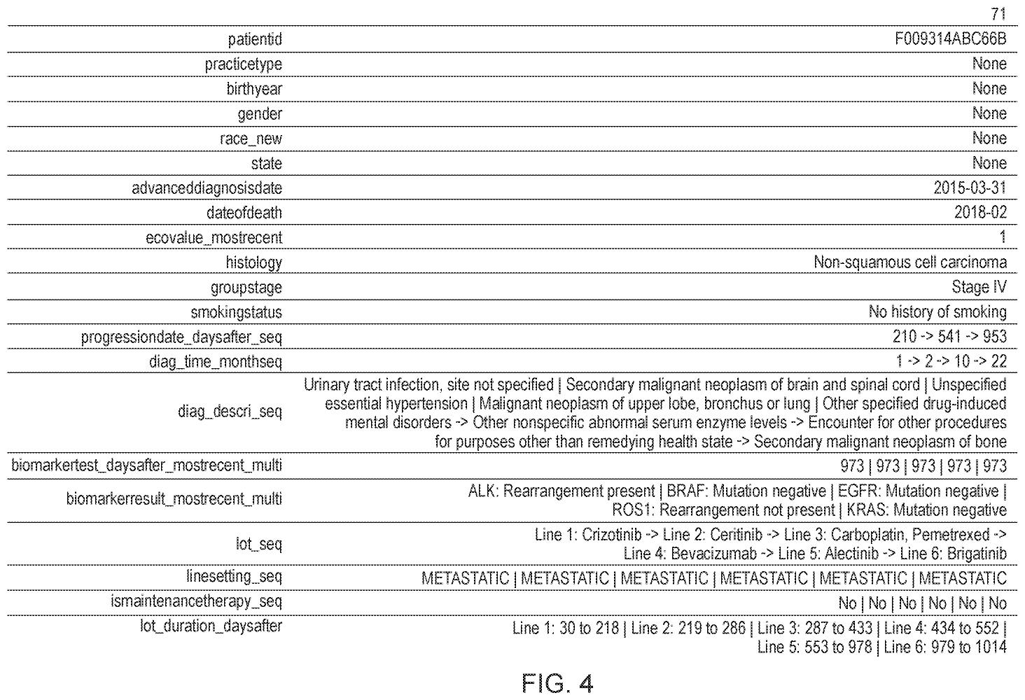

FIG. 5B shows two examples of how a decision-tree query may relate to a temporal sequence that would require derivation using the subject data or that may be inaccessible using the subject data. For example, the left table represents a decision-tree query pertaining to a time at which a biomarker test (to detect an estimated Glomerular Filtration Rate) was administered relative to a time at which a first-line treatment was received. In this case, the date of the biomarker test was available in the subject data, as was the date at which the first-line treatment commenced. While the subject data may lack an explicit identification of time difference between events or an order in which events occurred, this information can be important to evaluate decision-tree queries. For example, as illustrated in the depicted right table, a temporal relation query in a decision tree can be based on whether “Progression” occurred on first line therapy or subsequent therapy. As another example, a query can be based on whether “Progression” occurred on a particular compound “osimertinib”. However, the subject data may provide dates such as date of progression “2018 Jun. 30”. Logics and math transformation can then be performed standardize the subject data against guideline queries. As yet another example, death in the subject data also marks a progression event and date of death can be treated as date of progression and derived with temporal relation as a consequence.

FIG. 5C shows an example where the decision tree query relates to a quantitative metric. However, the test results that were included in the subject data includes measurements and ranges that are capped at values differing from thresholds specified in the decision tree. Further, the subject data includes categorical test results (e.g., unsuccessful/indeterminate test) and results of a different granularity than those that are to be evaluated by nodes on a decision tree. Thus, to evaluate the query, logic would need to be implemented to indicate how to handle a circumstance where a threshold specified in the decision tree is within a range identified in the subject data.

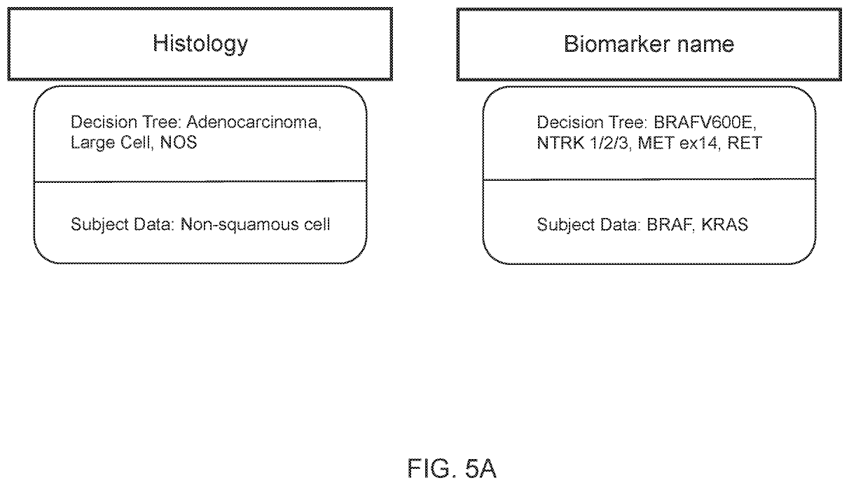

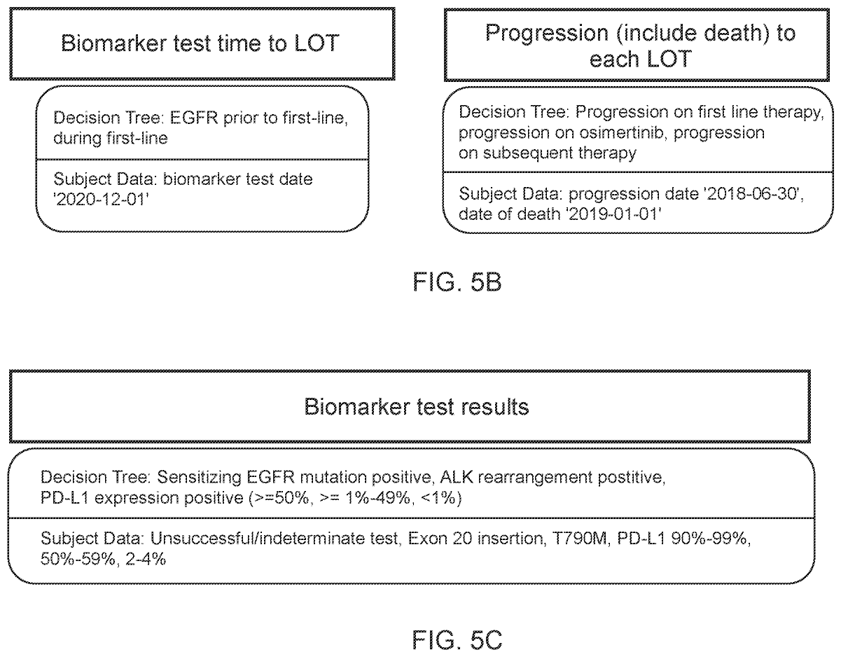

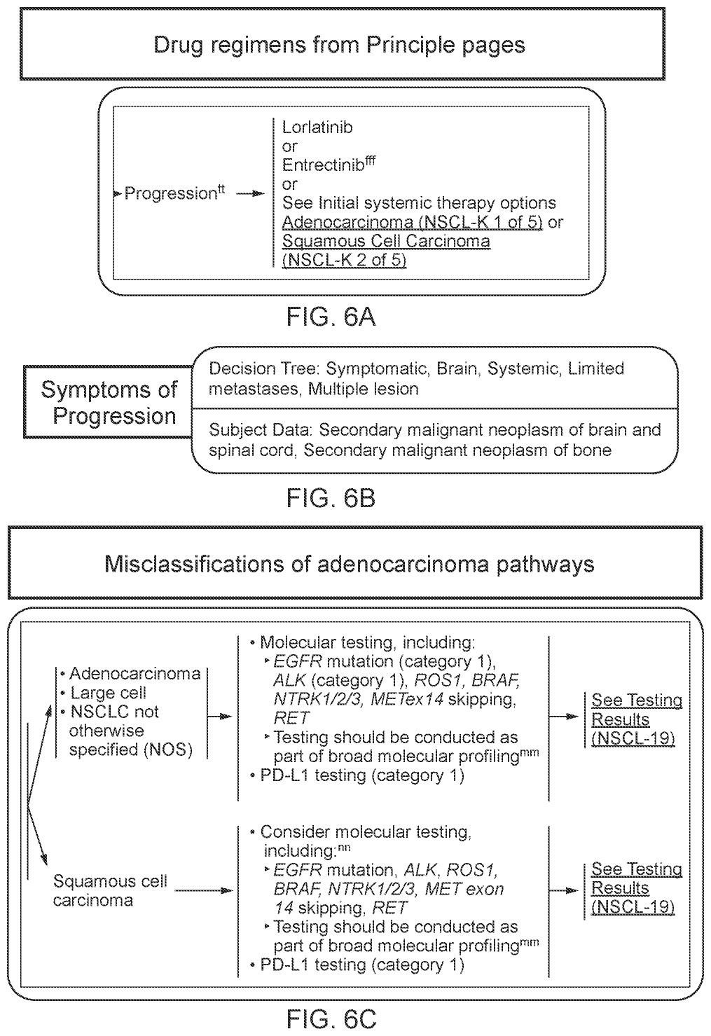

Each of FIGS. 6A-6B illustrate a scenario of mapping subject data to values pertaining to a query in decision tree. FIG. 6A illustrates a node in a decision tree referring to (linking to) two Principle pages, NSCLC-K 1 of 5 and NSCLC-K 2 of 5. The two Principle pages include content that was curated and fed into leaf-node-specific data sets. The two Principle pages (not shown) include information of drug protocol to be used. If the hyperlinked phrases themselves were used, the information in the linked pages would be lost. Therefore, instead of treating NSCLC-K 1 of 5 and NSCLC-K 2 of 5 literally, the text information from the page of NSCLC-K 1 of 5 and NSCLC-K 2 of 5 can be extracted and appended to a current version of the decision tree. In this way, the tokenizer controller is able to tokenize the information from these two pages for further steps and thus allow a better matching.

FIG. 6B illustrates the information corresponding to a structured data set and mapped information pertaining to transformed navigation to a decision tree. Here, a query identified in a decision tree relates to whether a subject has multiple lesions. This information is not specifically present in the subject data, though it may be inferred that the subject has multiple lesions given that the subject data indicates that there is a malignant neoplasm in each of three areas (brain, spinal cord, and bone). Another query identified in the decision tree is whether the metastases are “limited”. Again, the fact that the subject data identifies a neoplasm in different areas suggests that metastases has occurred. However, “limited” is not defined in the guideline. Thus, a mapping may define what qualifies as “limited” (e.g., as identifying a lower threshold for a number of body areas within which a neoplasm was detected).

As yet another exemplary challenge, as illustrated in FIG. 6C, keys in a pathway leading towards an identification of a particular treatment for adenocarcinoma may be very similar in relative frequencies to those in a pathway leading towards an identification of a particular treatment for squamous cell carcinoma (e.g., with a single key being different, i.e., “adenocarcinoma” versus “squamous”). Accordingly, subjects diagnosed with these histology subtypes could be assigned or classified into the wrong pathway, as the algorithm may confuse the two pathways if tokenization and vectorization are applied on the two pathways indiscriminately. To address this possibility, a weight (relative frequency) of the adenocarcinoma pathway was increased in the adenocarcinoma key in the adenocarcinoma pathway to facilitate discriminating between the two pathways.

### IV.B. Example 2

Structured data sets for each of 36,469 subjects who had aggressive non-small cell lung cancer was accessed. The subject data included biomarker test data indicating whether any of 6 mutations was available for 55% (n=20,093) of the subjects and was unavailable for 45% (n=16,365) of the subjects.

For each subject, the subject data was converted to structured subject data including a first set of key-value pairs. The structured subject data was then converted into a second set of key-value pairs, where keys in the second set of key-value pairs were different than keys in the first set of key-value pairs. The keys in the second set of key-value pairs were selected by identifying terms in decision trees (where the terms are used to define queries at decision nodes) that are inconsistently used across multiple decision trees, such that data indicating relative frequencies of terms (term frequencies scaled by inverse trajectory frequencies) inform selection of the keys. Specifically, n-gram tokenization was used to identify keys or tokens (e.g., one-word, two-word, and three-word tokens). The tokens can then be projected into the multidimensional space, thereby converting a representation of the structured data into a numeric vector.

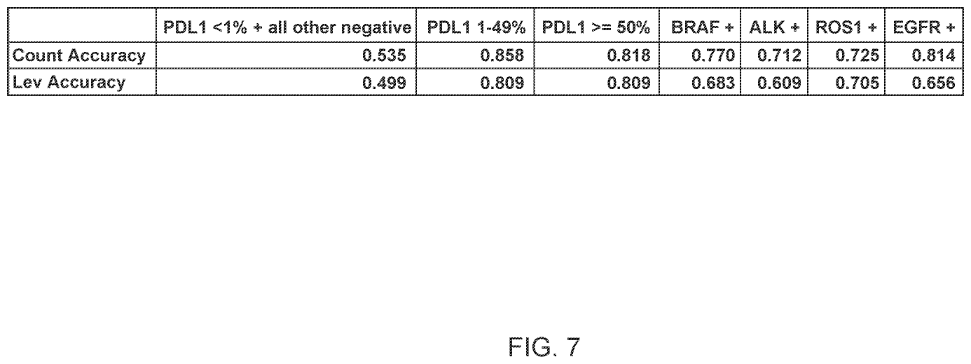

FIG. 7 shows the preliminary evaluations of the performance of the algorithm on matching a given subject to correct decision-trajectory. Two metrics are chosen to represent the performance. A first accuracy metric is based on the number of nodes in the guideline path that are correctly matched. For example, if a subject's true decision-tree trajectory consists of four nodes: A->B->C->D and the prediction is A->F->D, the accuracy metric is Ace=⅔=0.666. This reflects that two predicted nodes are among the true paths, and the length of prediction was used as the denominator, which is 3.

A second accuracy metric is based on Levenshtein distance, which is the minimum number of single edits (insertions, deletions or substitutions) required to change one sequence representing a true trajectory that applies to a subject into a trajectory predicted as applying to the subject. For example, if a subject's true decision-tree trajectory consists of four nodes: A->B->C->D and the prediction is A->F->D, the accuracy metric is Ace=1−¾=0.25. It means that at least three operations are required to convert the predicted trajectory into the true trajectory: deleting F, adding B, and adding C. The 4 indicates the length of ground truth, and it is the longer one between ground truth and prediction to be used. The fraction (¾) is then subtracted from 1 to generate the accuracy score.

To derive the accuracy scores in FIG. 7, trajectories were manually identified for 35 subjects (5 subjects in 7 categories). The categories were selected to reflect the distribution of subjects on biomarker testing results. Then both types of the aforementioned accuracy scores were calculated and averaged among the 5 subjects for that category to generate the numbers as shown in the depicted charts.

The scores themselves are not meant to be compared across existing algorithms as the testing case here may lack of generalizability. However, the scores can be useful for internal quality control purposes, such that performance of algorithm can be monitored during development. Further, the scores can be evaluated to infer for which category of subjects the algorithm performs better or worse.”

The first header row in the table shown in FIG. 7 identifies select keys in the second set of key-value pairs. Each of these keys can be associated with a “Yes” or “No” label to indicate whether the biomarker was detected. The numbers in the second row identify, for each biomarker, an average count accuracy score of predicted trajectories across five subjects in that category. The accuracy score was calculated by assuming that manually constructed trajectories are true trajectories. The numbers in the third row identify the average Levenshtein accuracy score across five subjects in that category (again based on a comparison with corresponding manually identified trajectory).

### V. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method comprising:
accessing a structured data set that includes a first set of key-value pairs, each of the first set of key-value pairs characterizing an assessment result or protocol characteristic for a subject;
transforming the first set of key-value pairs into a second set of key-value pairs, wherein at least some keys in the second set of key-value pairs are different from each key in the first set of key-value pairs;
projecting the second set of key-value pairs to identify a subject-specific point within a multi-dimensional space;
accessing one or more decision trees that include a plurality of decision nodes and a plurality of leaf nodes, wherein each of the plurality of leaf nodes is connected to a root node via a leaf-node-specific trajectory, and wherein each of the plurality of decision nodes corresponds to a criterion based on at least one value in the second set of key-value pairs;
determining, for each leaf node in the one or more decision trees, a leaf-node-specific point within the multi-dimensional space based on the leaf-node-specific trajectory;
determining, for each leaf node in the one or more decision trees, a similarity score based on the leaf-node-specific point and the subject-specific point;
identifying an incomplete subset of the plurality of leaf nodes based on the similarity scores;
retrieving state or protocol information associated with each leaf node in the incomplete subset; and
generating an output associated with the subject that includes the state or protocol information.

2. The computer-implemented method of claim 1, wherein, for each leaf node in the one or more decision trees, determining the leaf-node specific point includes:
transforming the leaf-node-specific trajectory into a first leaf-node-specific data set using text extraction;
transforming the first leaf-node-specific data set into a leaf-node-specific set of key-value pairs; and
projecting the second leaf-node-specific set of key-value pairs to identify the leaf-node-specific point.

3. The computer-implemented method of claim 1, wherein determining the similarity score includes applying a cosine similarity function.

4. The computer-implemented method of claim 1, further comprising: determining, for each term of a set of terms, an inverse trajectory frequency
that indicates how frequently the term occurs across leaf-node-specific trajectories associated with leaf nodes in the one or more decision trees; and
determining, for each term of the set of terms, a term frequency that indicates how frequently the term occurs in each of the leaf-node-specific trajectories;
wherein values in the second set of key-value pairs are defined based on the determined inverse trajectory frequencies and the term frequencies.

5. The computer-implemented method of claim 1, wherein the output further includes, for each node in the incomplete subset, queries represented by decision nodes in the leaf-node-specific trajectory.

6. The computer-implemented method of claim 1, wherein the output includes protocol information that identifies a potential treatment for the subject.

7. The computer-implemented method of claim 1, wherein the structured data set includes, for each of the first set of key-value pairs, an initial timestamp, and wherein transforming the first set of key-value pairs into a second set of key-value pairs includes:
classifying a particular key-value pair of the first set of key-value pairs as an indexing event;
generating, for each key-value pair of the first set of key-value pairs, a modified timestamp using the initial timestamp associated with the key-value pair and the initial timestamp associated with the particular key-value pair;
detecting that a decision node in the one or more decision trees includes a query to determine whether a particular event occurred within a particular time period relative to occurrence of another particular event, wherein the other particular event corresponds to the indexing event;
performing a query to determine whether the first set of key-value pairs includes a first particular key-value pair that is representative of the particular event and that is associated with a modified timestamp within the particular time range; and
defining a second particular key-value pair based on a result of the query, wherein the second set of key-value pairs includes the second key-value pair.

8. A computer-program product tangibly embodied in a non-transitory machine-readable storage medium, including instructions configured to cause one or more data processors to perform a set of actions including:
accessing a structured data set that includes a first set of key-value pairs, each of the first set of key-value pairs characterizing an assessment result or protocol characteristic for a subject;
transforming the first set of key-value pairs into a second set of key-value pairs, wherein at least some keys in the second set of key-value pairs are different from each key in the first set of key-value pairs;
projecting the second set of key-value pairs to identify a subject-specific point within a multi-dimensional space;
accessing one or more decision trees that include a plurality of decision nodes and a plurality of leaf nodes, wherein each of the plurality of leaf nodes is connected to a root node via a leaf-node-specific trajectory, and wherein each of the plurality of decision nodes corresponds to a criterion based on at least one value in the second set of key-value pairs;
determining, for each leaf node in the one or more decision trees, a leaf-node-specific point within the multi-dimensional space based on the leaf-node-specific trajectory;
determining, for each leaf node in the one or more decision trees, a similarity score based on the leaf-node-specific point and the subject-specific point;
identifying an incomplete subset of the plurality of leaf nodes based on the similarity scores;
retrieving state or protocol information associated with each leaf node in the incomplete subset; and
generating an output associated with the subject that includes the state or protocol information.

9. The computer-program product of claim 8, wherein, for each leaf node in the one or more decision trees, determining the leaf-node specific point includes:
transforming the leaf-node-specific trajectory into a first leaf-node-specific data set using text extraction;
transforming the first leaf-node-specific data set into a leaf-node-specific set of key-value pairs; and
projecting the second leaf-node-specific set of key-value pairs to identify the leaf-node-specific point.

10. The computer-program product of claim 8, wherein determining the similarity score includes applying a cosine similarity function.

11. The computer-program product of claim 8, wherein the set of actions further includes:
determining, for each term of a set of terms, an inverse trajectory frequency that indicates how frequently the term occurs across leaf-node-specific trajectories associated with leaf nodes in the one or more decision trees; and
determining, for each term of the set of terms, a term frequency that indicates how frequently the term occurs in each of the leaf-node-specific trajectories;
wherein values in the second set of key-value pairs are defined based on the determined inverse trajectory frequencies and the term frequencies.

12. The computer-program product of claim 8, wherein the output further includes, for each node in the incomplete subset, queries represented by decision nodes in the leaf-node-specific trajectory.

13. The computer-program product of claim 8, wherein the output includes protocol information that identifies a potential treatment for the subject.

14. The computer-program product of claim 8, wherein the structured data set includes, for each of the first set of key-value pairs, an initial timestamp, and wherein transforming the first set of key-value pairs into a second set of key-value pairs includes:
classifying a particular key-value pair of the first set of key-value pairs as an indexing event;
generating, for each key-value pair of the first set of key-value pairs, a modified timestamp using the initial timestamp associated with the key-value pair and the initial timestamp associated with the particular key-value pair;
detecting that a decision node in the one or more decision trees includes a query to determine whether a particular event occurred within a particular time period relative to occurrence of another particular event, wherein the other particular event corresponds to the indexing event;
performing a query to determine whether the first set of key-value pairs includes a first particular key-value pair that is representative of the particular event and that is associated with a modified timestamp within the particular time range; and
defining a second particular key-value pair based on a result of the query, wherein the second set of key-value pairs includes the second key-value pair.

15. A system comprising:
one or more data processors; and
a non-transitory computer readable storage medium containing instructions which, when executed on the one or more data processors, cause the one or more data processors to perform a set of actions including:
accessing a structured data set that includes a first set of key-value pairs, each of the first set of key-value pairs characterizing an assessment result or protocol characteristic for a subject;
transforming the first set of key-value pairs into a second set of key-value pairs, wherein at least some keys in the second set of key-value pairs are different from each key in the first set of key-value pairs;
projecting the second set of key-value pairs to identify a subject-specific point within a multi-dimensional space;
accessing one or more decision trees that include a plurality of decision nodes and a plurality of leaf nodes, wherein each of the plurality of leaf nodes is connected to a root node via a leaf-node-specific trajectory, and wherein each of the plurality of decision nodes corresponds to a criterion based on at least one value in the second set of key-value pairs;
determining, for each leaf node in the one or more decision trees, a leaf-node-specific point within the multi-dimensional space based on the leaf-node-specific trajectory;
determining, for each leaf node in the one or more decision trees, a similarity score based on the leaf-node-specific point and the subject-specific point;
identifying an incomplete subset of the plurality of leaf nodes based on the similarity scores;
retrieving state or protocol information associated with each leaf node in the incomplete subset; and
generating an output associated with the subject that includes the state or protocol information.

16. The system of claim 15, wherein, for each leaf node in the one or more decision trees, determining the leaf-node specific point includes:
transforming the leaf-node-specific trajectory into a first leaf-node-specific data set using text extraction;
transforming the first leaf-node-specific data set into a leaf-node-specific set of key-value pairs; and
projecting the second leaf-node-specific set of key-value pairs to identify the leaf-node-specific point.

17. The system of claim 15, wherein determining the similarity score includes applying a cosine similarity function.

18. The system of claim 15, wherein the set of actions further includes: determining, for each term of a set of terms, an inverse trajectory frequency
that indicates how frequently the term occurs across leaf-node-specific trajectories associated with leaf nodes in the one or more decision trees; and
determining, for each term of the set of terms, a term frequency that indicates how frequently the term occurs in each of the leaf-node-specific trajectories;
wherein values in the second set of key-value pairs are defined based on the determined inverse trajectory frequencies and the term frequencies.

19. The system of claim 15, wherein the output further includes, for each node in the incomplete subset, queries represented by decision nodes in the leaf-node-specific trajectory.

20. The system of claim 15, wherein the output includes protocol information that identifies a potential treatment for the subject.
